(19) United States
(12) Patent Application Publication
(10) Pub. No.: US 2026/0314692 A1
(43) Pub. Date: Oct. 15, 2026

(54) AEROSOL-GENERATING DEVICE WITH IMPROVED AIRFLOW

(71) Applicant: Alexza Pharmaceuticals, Inc., Fremont, CA (US)

(72) Inventors: Amit MHATRE, Fremont, CA (US); Kenneth CHIH-PING CHANG, Fremont, CA (US)

(73) Assignee: Alexza Pharmaceuticals, Inc., Fremont, CA (US)

(21) Appl. No.: 19/485,373

(22) PCT Filed: Jun. 14, 2024
(86) PCT No.: PCT/US2024/033966

§ 371 Date: Nov. 18, 2025

Related U.S. Application Data
(60) Provisional application No. 63/521,083, filed on Jun. 14, 2023.

(51) Int. Cl. A24F 40/485; A24F 40/20; A24F 40/42; A24F 40/51; A24F 40/70; A24F 47/00; A61K 31/404; A61K 31/4178; A61K 31/428; A61K 31/439; A61K 31/4468; A61K 31/465; A61K 31/473; A61K 31/48; A61K 31/519; A61K 31/5517; A61K 31/553; A61M 15/00

(52) U.S. Cl. CPC A24F 40/485; A24F 40/42; A24F 40/51; A24F 40/70; A24F 47/00; A61K 31/404; A61K 31/4178; A61K 31/428; A61K 31/439; A61K 31/4468; A61K 31/465; A61K 31/473; A61K 31/48; A61K 31/519; A61K 31/5517; A61K 31/553; A61M 15/0066; A24F 40/20; A61M 2205/127; A61M 2205/273

(57) ABSTRACT

An aerosol-generating device including top and bottom molded parts assembled to form an airflow chamber extending between an air inlet and an air outlet, in the form of a mouthpiece, the air chamber including a solid drug film coated on at least one foil substrate and a plurality of airflow control features arranged throughout the inner walls of the airflow chamber.

### FIELD

The embodiments relate to the field of aerosol drug delivery devices, and to the field of devices having enhanced airflow control for the precise administration of condensation aerosols.

### BACKGROUND

Existing drug condensation aerosol delivery devices, such as electronic cigarettes, often use heated liquids or plant products to generate aerosols for inhalation. However, these devices are not suitable for the precise dosing required in medical applications. Precise dosing in drug aerosol delivery devices is essential for consistency and efficacy. This is typically achieved by accurately depositing a measured dose of a drug onto a substrate that is then heated to form an aerosol.

Several prior art references, including WO2019152873, WO2017189883, and WO2016145075, describe methods for generating condensation aerosols, controlling aerosol particle size distribution (aPSD), and ensuring precise drug delivery. These references are incorporated by reference for their detailed descriptions of condensation aerosol formation, vaporization temperature control, and substrate design. The consistency of the emitted dose (ED) from such devices is critical. To achieve this, controlling the airflow throughout the substrate during vaporization is crucial. However, conventional designs often face issues with uneven heating and cooling, leading to inconsistent aerosol production.

Thus, there remains a need for a device that can deliver a reproducible and accurate dose of drug aerosol with high purity, increased reproducibility, and improved therapy.

### SUMMARY

An aerosol-generating device for generating a drug condensation aerosol by thermal vaporization of the drug. Further, the embodiments relate to an aerosol-generating device including airflow control features to provide a controllable and uniform flow of air throughout a foil substrate, coated with a drug film susceptible of producing aerosolized particles, to achieve a uniform cooling effect and drug condensation for optimal aerosol delivery.

To this end, the airflow control features include a perforated bulkhead having a plurality of through-holes to control the amount of airflow allowed to enter the device; such resistance may be tuned to meet the needs of a particular application by varying the number, size, pattern and/or spacing of said through-holes throughout the bulkhead.

The perforated bulkhead also acts as a safety filter to prevent particles larger than 1 mm from entering the main flow area.

Further, the aerosol-generating device also includes a selection of one or more additional airflow control features, i.e., a set of meshed teeth, a plurality of transverse frets, a plurality of gap channels, airflow control elements, to add extra airflow resistance and/or allow control over turbulent flow and/or generate distinct airflow paths for a uniform cooling effect and/or allow control over possible air leakage.

It is also envisaged that the aerosol-generating device is designed as a disposable cartridge that is further equipped with means for connecting the device to a handheld controller to electrically enable vaporization of the drug within the solid drug film, enabling the electrical vaporization of the drug film.

In summary, the handheld medical device described herein offers an innovative solution for generating drug condensation aerosols through thermal vaporization. The device's unique combination of airflow control features ensures improved aerosol delivery while maintaining efficiency and control. The disclosed embodiments also provide compatibility with a handheld controller.

### BRIEF DESCRIPTION OF THE DRAWINGS

FIG. 1 is a perspective view of the aerosol-generating device according to a first embodiment.

FIG. 2 is an exploded view of the aerosol-generating device of FIG. 1

FIG. 3 is a cross-section view of the device of FIG. 1

FIG. 4 is a top plan view of the top molded part of the device of FIG. 1.

FIG. 5 is a top plan view of the bottom molded part of the device of FIG. 1.

FIG. 6 is a top plan view of the top molded part according of the device of FIG. 1 having additional airflow control features.

FIG. 7 is a top plan view of the bottom molded part according of the device of FIG. 1 having additional airflow control features.

FIG. 8 is a front view of the bottom molded part of FIG. 7.

FIG. 9 is a perspective view of the bottom molded part of FIG. 7.

FIG. 10 is a front view of the assembled top and bottom parts of FIGS. 6 and 7.

FIG. 11 shows thermal images of the airflow patterns of an aerosol-generating device devoid of airflow control features.

FIG. 12 shows a computational simulation of the airflow fluid dynamics within an aerosol-generating device including the airflow control features according to the first embodiment.

FIG. 13 is an exploded view of an aerosol-generating device according to a second embodiment.

FIG. 14 is a top plan view of the device of FIG. 13.

FIG. 15 is a perspective cross-section view of a device according to a form of the embodiment of FIG. 13.

FIG. 16 is a perspective cross-section view of a device according to a further form of the embodiment FIG. 13.

FIG. 17 shows a computational simulation of the airflow fluid dynamics within a device having a bulkhead assembly configuration according to the device of FIG. 16.

FIG. 18 shows thermal images of the airflow patterns obtained at different times during heating of the foil substrate of a device according to the second embodiment.

FIG. 19 shows thermal images of the airflow patterns obtained at different times during heating of the foil substrate of a device according to another form of the second embodiment.

FIG. 20 shows thermal images of the airflow patterns at different times during heating of the foil substrate of a device including a perforated bulkhead, without airflow directing features, arranged at the air inlet.

FIG. 21 shows a perspective view of a device and a handheld controller according to an embodiment.

### DETAILED DESCRIPTION

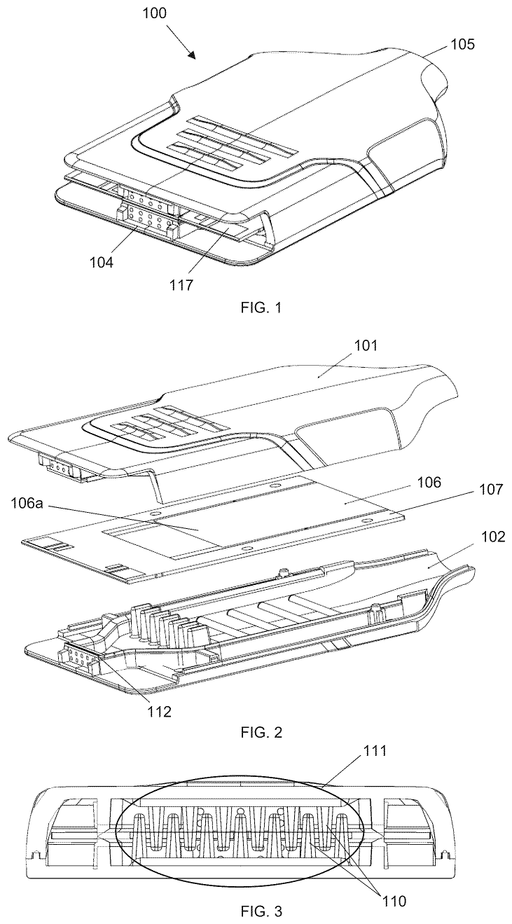

FIG. 1 shows an aerosol-generating device 100 in accordance with a first embodiment. The device 100, as shown in FIG. 2, includes a top molded part 101 and a bottom molded part 102 which are assembled to form an airflow chamber 103 extending between an air inlet 104 located at a distal end thereof, and an air outlet 105, in the form of a mouthpiece, at a proximal end thereof.

The airflow chamber 103 is defined by inner walls 101a, 101b, 101c, 102a, 102b, 102c.

In other words, the device 100 can be manufactured in two molded parts 101, 102, which facilitates the formation of features on the inner walls of the airflow chamber 103.

The top and bottom molded parts 101, 102 may be assembled mechanically, i.e., press-fitting, slip-fitting, or snap-fitting. Alternatively, assembly may also be performed by application of energy to weld components together, i.e., welding, or by means of solvent bonding.

In a particular form of the present embodiment, the molded parts 101, 102 are assembled mechanically by snap-fitting.

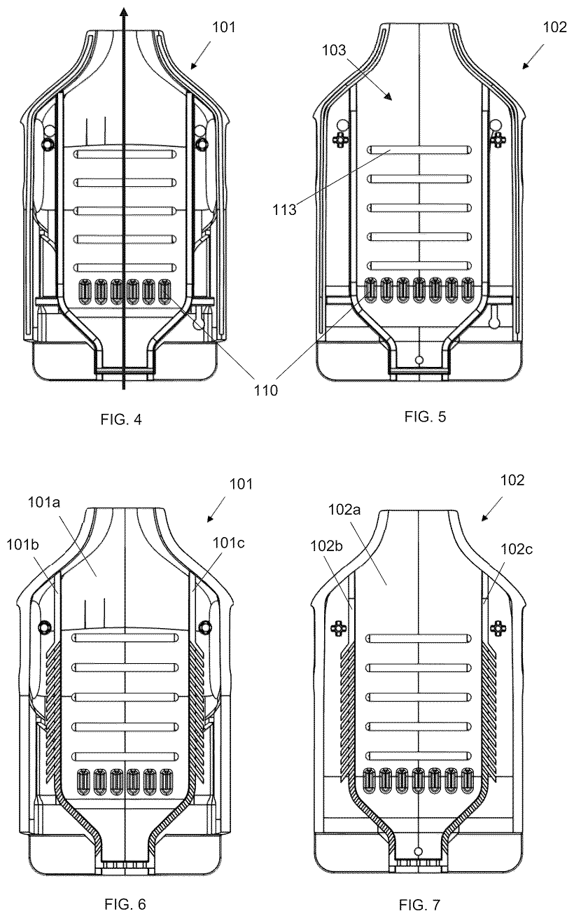

The combination of the air inlet 104, airflow chamber 103, and air outlet 105 can be considered to form or represent the primary airflow path of the device 100, whereby the airflow resulting from a user inhalation travels in the general direction indicated by the single-headed arrow in FIG. 4, from the air inlet 104 (downstream) to the air outlet 105 (upstream).

The airflow chamber 103 includes a solid drug film coated on at least one foil substrate 106. The at least one foil substrate 106 can have a surface with or without perforations.

In a form of the first embodiment, the airflow chamber 103 includes a plurality of foil substrates 106 capable of being heated independently to enable multiple doses within a single device.

In a particular form, the at least one foil substrate 106 is equidistantly supported, within the air chamber 103, from the top and bottom molded parts 101, 102 by a solid support 107.

In a particular form, the solid support is a printed circuit board (PCB) assembly 107, as shown in FIG. 2, and the top and bottom molded parts 101, 102 are assembled against the PCB assembly 107.

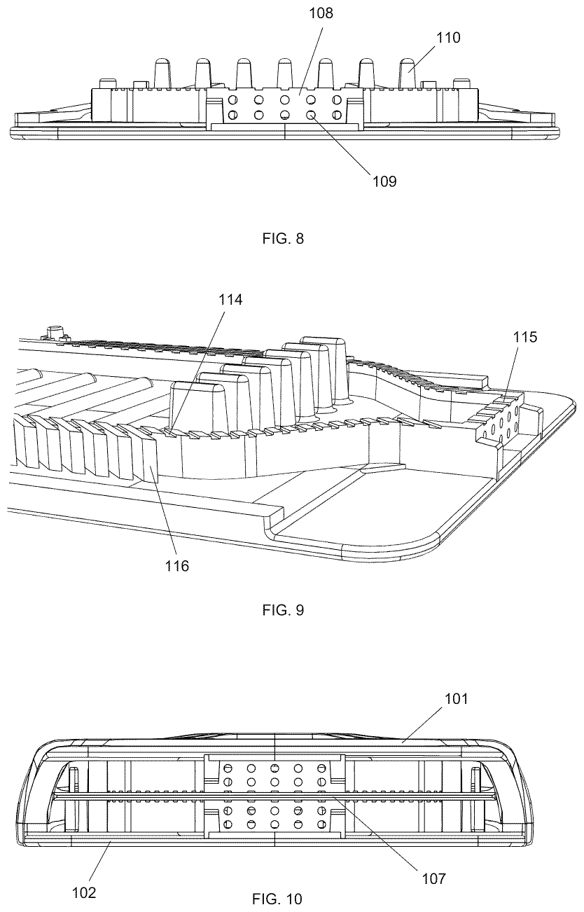

The top and bottom molded parts 101, 102 of the aerosol-generating device 100, each, include airflow control features in the form of a perforated bulkhead 108, having a plurality of through-holes 109, said bulkhead 108 being arranged flush with or adjacent to the air inlet 104 portion of each of the parts 101, 102, as shown in FIG. 8, and substantially perpendicular to the air inlet 104 to air outlet 105 axis, as shown in FIG. 8 Said bulkhead 108 is provided to modify the resistance to draw air through the device 100 during a user inhalation.

The perforated bulkhead 108 additionally acts as a safety filter to prevent particles larger than 1 mm from passing through the plurality of through-holes 109.

The design of the through-holes 109 can vary in size, number, location, and pattern.

The diameter of the through-holes 109 is typically between 0.25 mm and 2 mm, particularly between 0.5 mm to 1.5 mm, and more particularly 1 mm.

The plurality of through-holes 109 may take any shape including elliptical, circular, oval, rectangular, square-shaped or of any desired polygonal form.

In a particular form, each bulkhead 108 has two sets of five through-holes 109, evenly spaced apart in the transverse direction of the bulkhead 108 and arranged one over the other.

As shown in FIGS. 4 and 5, at the distal end of the airflow chamber 103, there is a funnel-shaped portion with a narrower end corresponding to the air inlet 104. The airflow chamber 103 widens at the other end of the funnel-shaped portion into a substantially rectangular-shaped portion.

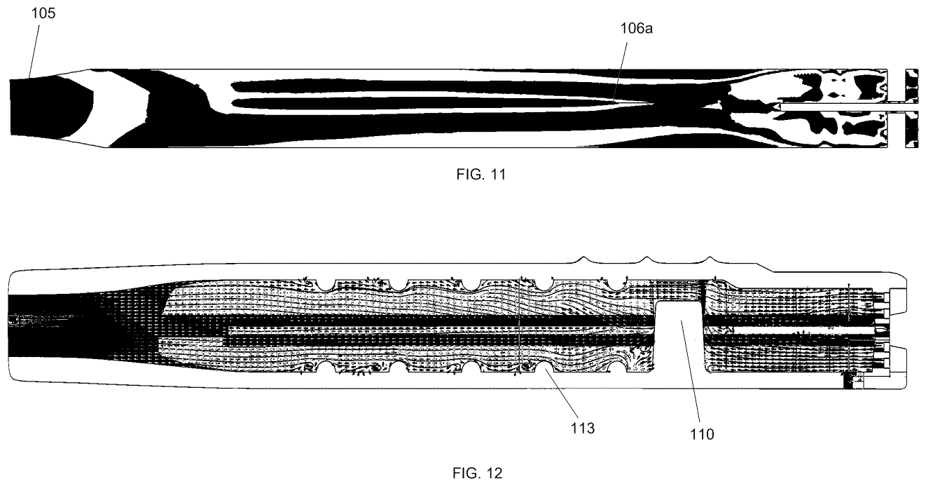

As shown in FIG. 11, showing a thermal image of the device devoid of airflow control features, the flow of air tends to naturally concentrate, due to flow separation, when the flow area widens towards the substantially rectangular-shaped portion, directly impacting the leading edge 106a of the at least one foil substrate 106 or the leading foil substrate, i.e., the foil substrate 106 closest to the air inlet 104, in the case where there is more than one foil substrate 106, thereby resulting in uneven cooling.

As used herein, the term “flow area” refers to the cross-sectional area of the airflow chamber 103 in a plane that is perpendicular to the general direction of the air flow through the airflow chamber 103.

To address this issue, the top and bottom molded parts 101, 102 of the aerosol-generating device 100, include additional airflow control features in the form of a plurality of interspaced teeth 110, as shown in FIGS. 4 and 5, arranged upstream of the perforated bulkhead 108. Said interspaced teeth 110 add additional airflow resistance and direct flow away from the leading edge 106a of the foil substrate 106, as shown in FIG. 12.

The top and bottom molded parts 101, 102 are assembled in such a manner that the two parts 101, 102 form an airflow chamber 103 including a set of meshed teeth 111 having air gaps between the interspaced teeth 110, as shown in FIG. 3.

The set of meshed teeth 111 allows airflow to be directed essentially over and under the foil substrate 106 to avoid air from concentrating directly onto the leading edge 106a of the foil substrate 106. To this regard, the air gap at the ends of the teeth tips is larger than the air gap between consecutive teeth.

The design, number, size, and location of the interspaced teeth 110 may vary.

Said plurality of interspaced teeth 110 can have different designs, including, but not limited to, fin-like, square, teardrop, and oval.

In a particular form, the plurality of interspaced teeth 110 have an oval design.

In a particular form, the top molded part 101 includes six interspaced teeth 110 and the bottom molded part 102 includes seven interspaced teeth 110.

In a particular form, the distance between each consecutive interspaced teeth 110 of each molded part 101, 102 ranges from 0.5 mm to 2.5 mm, more particularly from 1 mm to 2 mm.

In a particular form, the height of each of said plurality of interspaced teeth 110 ranges from 5 mm to 8 mm, more particularly from 6 mm to 7 mm.

Such forms have been found to be particularly effective at preventing air from concentrating on the leading edge 106a of the foil substrate 106.

In a particular form of the first embodiment, the top and bottom parts 101, 102 further include additional airflow control features in the form of a plurality of transverse frets 113, as shown in FIG. 5, arranged throughout the inner walls 101a, 102a of the top and bottom parts 101, 102, respectively.

Said plurality of transverse frets 113 extend substantially perpendicular to the air inlet 104 to air outlet 105 axis and are arranged upstream the plurality of interspaced teeth 110 at spaced intervals to help air slow and generate vortices which direct air towards the foil substrate 106, while not affecting laminar flow around the foil substrate 106.

The design, size, number, and location of said plurality of transverse frets 113 may vary.

In a particular form, the top and bottom molded parts 101, 102, each, include five transverse frets 113 each.

In a particular form, the length of the transverse frets 113 ranges from 25 mm to 30 mm, more preferably from 27 mm to 28 mm.

Such forms have been found to be particularly effective at directing more air toward the at least one foil substrate 106.

In a form of the first embodiment, as shown in FIG. 2, the top and bottom parts 101, 102 include an elastic gasket 112, to form a sealed airflow chamber 103. Gasket geometry and material are designed to reduce the compression force needed during assembly.

In another form of the first embodiment, the top and bottom parts 101, 102 do not include the elastic gasket 112.

The top and bottom molded parts 101, 102 may, then, as shown in FIGS. 6 and 7, further includes additional airflow control features to allow air to leak in a controlled manner.

Said additional airflow control features, as shown in FIG. 9, include:
a plurality of first air channels 114, extending along at least part of the length of the side inner walls 101b, 101c, 102b, 102c; and/or
a plurality of second air channels 115, arranged on the bulkhead 108, over the through-holes 109.

In a particular form, said plurality of first air channels 114 are angled with respect to the air inlet 104 to air outlet 105 axis.

In a particular form, at least part of said plurality of first air channels 114 include guiding walls 116, as shown in FIG. 9.

The plurality of first and second air channels 114, 115 are added throughout at least part of the length of the side internal walls 101a, 101b, 102a, 102b, and throughout the bulkhead 108, respectively, to allow air to leak in a controlled manner, with minimal effect on bulk air flow.

In a particular form, the length of the plurality of the first and second air channels 114, 115 ranges between 1 mm and 3 mm.

The effective length of the plurality of first air channels 114 can be increased towards the air outlet 105 by the guiding walls 116, such that, air flow will avoid biasing towards the first air channels 114 that are closer to the air outlet 102, i.e., shortest path, the path of least resistance. The increased effective length adds additional flow resistance, preventing uneven flow from the air channels.

In a particular form, the effective length of the plurality of air channels 114 is between 3 mm and 9 mm, and more particularly between 5 mm and 7 mm.

The plurality of first and second air channels 114, 115 are sized to be smaller than the plurality of through-holes 109 but larger than potential gaps formed due to the physical tolerance between the parts 101, 102. Airflow will bias towards larger openings, such that size differentials ensure that any air that would normally leak from tolerance mismatch will instead flow through the controlled first and second air channels 114, 115, while still allowing most of the air to flow through the plurality of through-holes 109.

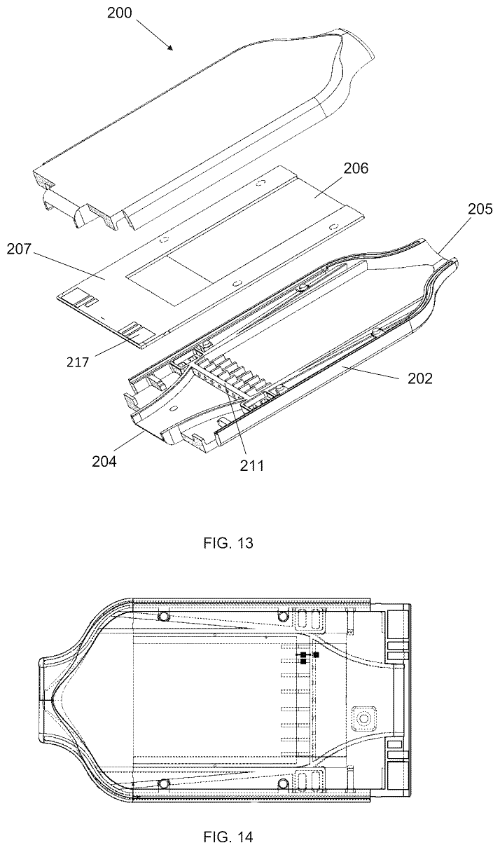

In a second embodiment, as shown in FIG. 13, the aerosol-generating device 200 includes a top molded part 201 and a bottom molded part 202 which are assembled to form an airflow chamber 203 extending between an air inlet 204 located at a distal end thereof, and an air outlet 205, in the form of a mouthpiece, at a proximal end thereof.

The airflow chamber 203 is defined by inner walls and includes a solid drug film coated on at least one foil substrate 206. The at least one foil substrate 206 can have a surface with or without perforations.

In a form of the second embodiment, the air chamber 203 includes a plurality of foil substrates 106 capable of being heated independently to enable multiple doses within a single device.

The at least one foil substrate 206 is supported, within the air chamber 203, by a solid support 207.

In a particular form, the solid support is a printed circuit board (PCB) assembly 207, as shown in FIG. 13, and the top and bottom molded parts 201, 202 are assembled against the PCB assembly 207.

The bottom molded part 201 includes airflow control features in the form of a bulkhead assembly 211, which includes a perforated bulkhead 208, having a plurality of through-holes 209, and a plurality of airflow directing elements 210 which lie flush with the proximal surface of the perforated bulkhead 208.

The plurality of through-holes 209 are oriented orthogonally to the plurality of airflow directing elements 210.

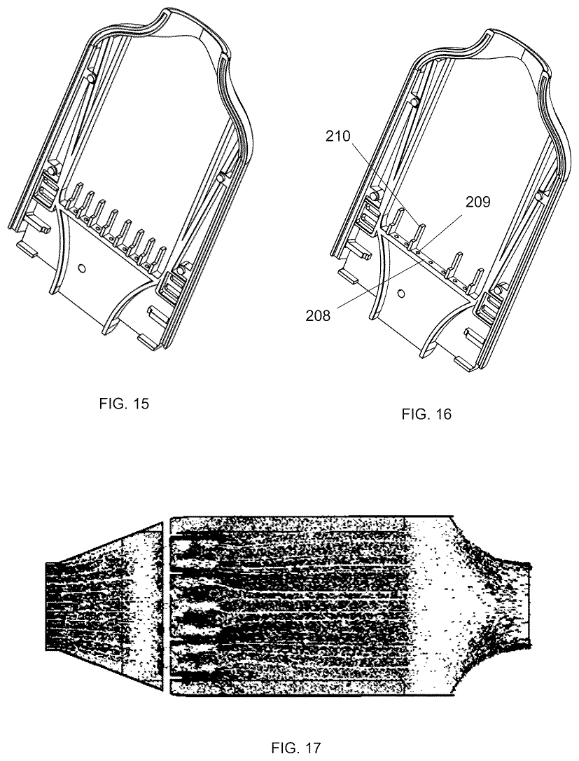

As shown in FIGS. 15 and 16, at the distal end of the airflow chamber 203, there is a funnel-shaped portion with a narrower end corresponding to the air inlet 204. The airflow chamber 203 widens at the other end of the funnel-shaped portion into a substantially rectangular-shaped portion.

In a particular form of the second embodiment, the bulkhead assembly 211 may be arranged at the interface between the funnel-shaped portion and the substantially rectangular-shaped portion of the airflow chamber 203.

The plurality of through-holes 209 may be arranged in a predetermined symmetrical pattern along the perforated bulkhead assembly 208.

In a form of the second embodiment, the plurality of through-holes 209 and/or the plurality of airflow directing elements 210 may be evenly spaced apart along the transverse direction of the bulkhead assembly 211. The airflow directing elements 210 may be arranged in the gap between each pair of continuous through-holes 209, as shown in FIG. 15, corresponding to a device 200 having a bulkhead assembly 211 including seven through-holes 209 and eight airflow directing elements 210.

In another form of the second embodiment, the plurality of through-holes 209 in the perforated bulkhead 208 can be arranged in sets of three, forming a triangular configuration 209a. Each set may be evenly spaced apart along the transverse direction of the perforated bulkhead 208.

The plurality of airflow directing elements 210 may also be arranged in gaps between each pair of three-hole sets 209a.

The plurality of through-holes 209 may take any shape including elliptical, circular, oval, rectangular, square-shaped or of any desired polygonal form.

In yet another form according to the second embodiment, the spacing of the plurality of through-holes 209 along the transverse direction of the perforated bulkhead 208 may not be even, but, for example, gradually become narrower from the middle portion towards both ends.

Similarly, the spacing of the airflow directing elements 210 may also vary, as shown in FIG. 16, corresponding to a device 200 including a bulkhead assembly 211 having seven through-holes 209, which are not evenly spaced apart along the perforated bulkhead 208 and four airflow directing elements 210.

The number of through-holes 209 in the perforated bulkhead 208 can range from three to fifteen, particularly from five to ten, and more particularly seven through-holes.

The diameter of the through-holes 209 is typically between 0.25 mm and 2 mm, particularly between 0.5 mm and 1.5 mm, and more particularly 1 mm.

The airflow directing elements 210 have a length of between 3 mm and 15 mm, with a specific value of 7 mm, and a width between 0.25 mm and 2 mm, more particularly between 0.5 mm and 1.5 mm.

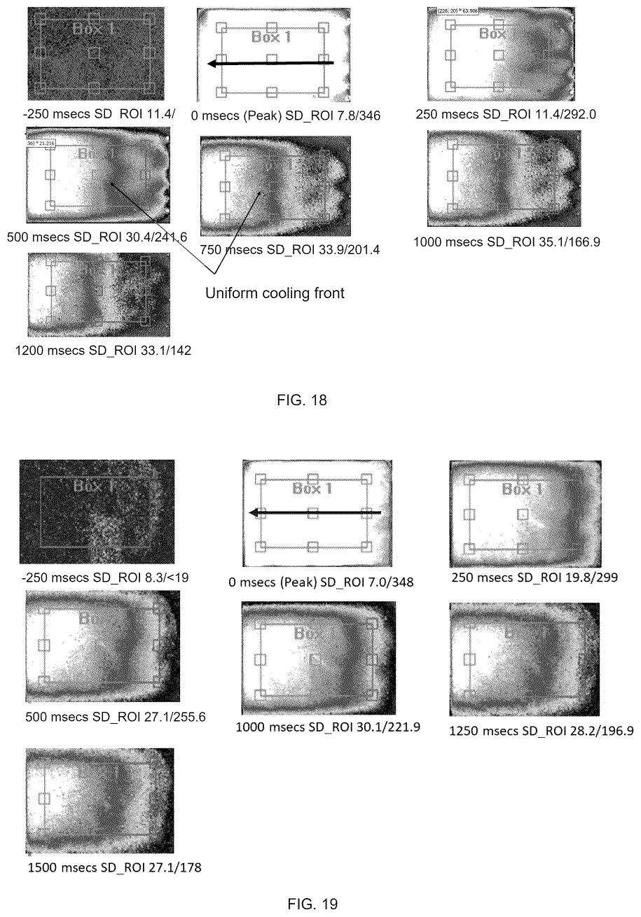

FIG. 18 shows airflow thermal images taken at different times during heating of the foil substrate 206 of a device 200 having a bulkhead assembly 211 including seven through-holes 209 and eight airflow directing elements 210, both evenly spaced along the transverse direction of the bulkhead assembly 211, each pair of continuous through-holes 209 having an airflow directing element 210 arranged in the gap therebetween, wherein the through-holes 209 have a diameter of 1 mm.

FIG. 19 shows airflow thermal images taken at different times during heating of the substrate foil 206 of a device 200 having a bulkhead with fifteen through-holes 209 arranged in sets of three 209a, having a triangular shape configuration, and four airflow directing elements 210, both evenly spaced along the transverse direction of the bulkhead, each pair of continuous set of three 209 having an airflow directing element 210 arranged in the gap therebetween, wherein the through-holes have a diameter of 0.7 mm.

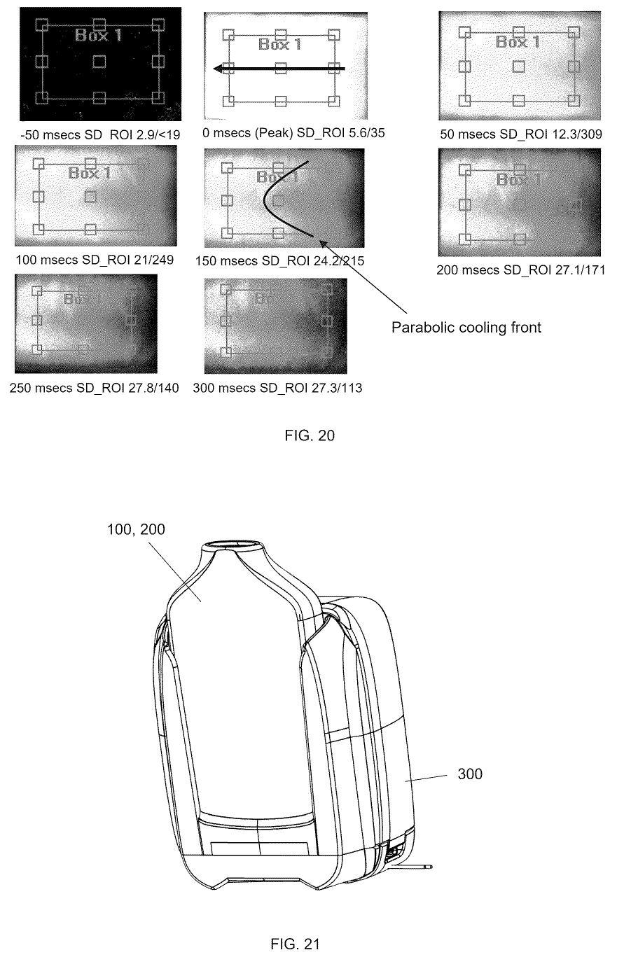

As may be inferred from FIGS. 18 and 19, both bulkhead assemblies 211 show the advancement of a uniform airflow front rather than the airflow front shown in FIG. 20, corresponding to a device including a perforated bulkhead devoid of airflow directing elements arranged at the narrower end of the funnel-shaped portion, just at the air inlet 204, wherein the images show an airflow front having a rather parabolic front.

FIG. 17 is a dynamic simulation showing airflow behavior wherein differentiated air flow streams are generated by the plurality of airflow directing elements 210 to provide a uniform airflow front trespassing the foil substrate 207 and generate a uniform cooling effect.

In a form of the second embodiment, the airflow directing elements 210 are L-shaped, with the long part of the L being substantially parallel to the air inlet 204 to air outlet 205 axis. The L-shaped airflow directing elements 210 may accommodate the foil substrate support 207, such that the long part of the L overhangs the surface of the foil substrate support 207.

According to the embodiments, the heating of the foil substrate 106, 206 can be achieved through electrical means or through a chemical heat pack based on an exothermic chemical reaction.

The device of the embodiments may be in the form of a disposable cartridge 100, 200 and include means for connection to a handheld controller 300, as shown in FIG. 21. These means can include one or more connectors 117, 217 to establish an electrical connection between the disposable cartridge 100, 200 and the handheld controller 300. The handheld controller 300 itself can have a heating circuit to vaporize the drug in the solid drug film.

In a form of the embodiments, the airflow chamber 103, 203 can incorporate antistatic material.

The device 100, 200 is intended for therapeutic use in various conditions or episodes, depending on the drug used in the solid drug film. The specified conditions include agitation, epilepsy, breakthrough pain, sleep disorders, Parkinson's disease, and nausea/vomiting.

While specific embodiments have been illustrated and described, it will be understood that various modifications can be made without departing from the scope of the embodiments.

### IR TEMPERATURE MEASUREMENT

The temperature was measured using FLIR Systems infrared cameras, Thermacam SC3000 and A655sc. The SC3000 infrared camera uses quantum well infrared photodetector technology, and the A655sc uses an uncooled microbolometer detector. These cameras are adequate for high sensitivity and accuracy and captures images up to 180 and 200 Hz, respectively. Temperature is calculated based on the amount of emitted infrared light. The camera was calibrated by heating the metal cylinders (resistive heating with a constant current DC power supply) to various steady-state temperatures between 200 and 400 ° C. and measuring the actual temperature with calibrated thermocouples (Omega, Stamford, CT).

### CFD MODELLING

Computational Fluid Dynamics (CFD) have been created using either Simulia® software under the 3DEXPERIENCE® platform version R2023x.HotFix0.10, or SolidWorks 2021 SP04.1 with Flow Simulation add-in. The CFD software uses a Navier-Stokes equations solver to solve the mathematical model including partial differential equations that describe the fluid flow. The CFD software solves the mathematical models with boundary conditions specific to our problem.

1-32. (canceled)

33. An aerosol-generating device for generating a drug condensation aerosol by thermal vaporization of the drug, said aerosol-generating device comprising:
a top molded part; and
a bottom molded part;
said top and bottom molded parts assembled to form an airflow chamber extending between an air inlet located at a distal end thereof, and an air outlet, in the form of a mouthpiece, at a proximal end thereof,
said airflow chamber comprising a solid drug film coated on an at least one foil substrate,
said top molded part and/or bottom molded part comprising airflow control features,
said airflow control features comprising a perforated bulkhead having a plurality of through-holes arranged flush with, or adjacent to, the air inlet portion of molded the parts, and a plurality of interspaced teeth arranged upstream of the perforated bulkhead; or
a bulkhead assembly comprising a perforated bulkhead, having a plurality of through-holes, and a plurality of airflow directing elements which lie flush with the proximal surface of the bulkhead, and wherein the plurality of through-holes is oriented orthogonally to the airflow directing elements.

34. The aerosol-generating device according to claim 33, wherein the plurality of interspaced teeth from the top and bottom parts form a set of meshed teeth having air gaps between the interspaced teeth.

35. The aerosol-generating device according to claim 33, wherein the top and bottom molded parts comprise additional airflow control features in the form of a plurality of longitudinal frets arranged on inner walls upstream the plurality of interspaced teeth.

36. The aerosol-generating device according to claim 33, wherein the top and bottom molded parts comprise an elastic gasket.

37. The aerosol-generating device according to claim 33, wherein the top and bottom molded parts do not include an elastic gasket.

38. The aerosol-generating device according to claim 37, wherein the top and bottom molded parts further comprise:
a plurality of first air channels extending along at least part of the length of inner walls of the top and bottom parts; and/or
a plurality of second air channels arranged throughout the bulkhead over the through-holes.

39. The aerosol-generating device according to claim 38, wherein the plurality of first air channels are angled with respect to the air inlet to air outlet axis.

40. The aerosol-generating device according to claim 38, wherein at least part of the plurality of first air channels comprises guiding walls.

41. The aerosol-generating device according to claim 38, wherein the effective length of the plurality of first air channels increases towards the air outlet.

42. The aerosol-generating device according to claim 33, wherein the plurality of airflow directing elements are L-shaped and the long part of the L is substantially parallel to the air inlet to air outlet axis.

43. The aerosol-generating device according to claim 42, wherein the long part of the L overhangs the surface of a foil substrate support.

44. The aerosol-generating device according to claim 33, wherein said device is in the form of a disposable cartridge and wherein the disposable cartridge further comprises means for connecting the device to a handheld controller.

45. Th aerosol-generating e device according to claim wherein said means for connecting the cartridge to the handheld controller include one or more connectors to electrically connect the disposable cartridge to the handheld controller.

46. The aerosol-generating device according to claim 33, wherein the airflow chamber comprises antistatic material.

47. The aerosol-generating device according to claim 33 for use in therapy, wherein the drug in the solid drug film is:
A) loxapine or its pharmaceutically acceptable salts, the condition or episode is agitation, comprising:
i. rapidly control mild to moderate agitation in adults with schizophrenia or bipolar disorder, or
ii. acute agitation associated with schizophrenia or bipolar disorder in adults;
B) alprazolam, estazolam or their pharmaceutically acceptable salts, the condition or episode is epilepsy, wherein epilepsy comprises seizures;
C) fentanyl or its pharmaceutically acceptable salts, the condition or episode is breakthrough pain;
D) zaleplon, almorexant or its pharmaceutically acceptable salts, the condition or episode is a sleep disorder comprising:
i. middle of the night awakening, or
ii. middle of the night insomnia;
E) apomorphine, pergolide, ropinirole, pramipexole, or its pharmaceutically acceptable salts, the condition or episode is Parkinson's disease, (including off-episodes in Parkinson's disease, and/or idiopathic Parkinson's disease);
F) granisetron, ondansetron, palonosetron or their pharmaceutically acceptable salts, the condition or episode is:
i. nausea,
ii. vomiting or
iii. cyclic vomiting syndrome;
G) nicotine or its pharmaceutically acceptable salts including nicotine meta-salicylate, the condition or episode is nicotine craving and/or effecting cessation of smoking; or
H) ropinirole, pramipexole, or rotigotine or their pharmaceutically acceptable salts, the condition or episode is restless legs syndrome.
